(19) United States
(12) Patent Application Publication
(10) Pub. No.: US 2006/0111659 A1
(43) Pub. Date: May 25, 2006

(54) Systems and methods for CSF drainage

(76) Inventor: Jonathan Tyler, Edmonton (CA)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/217,966

(22) Filed: Aug. 31, 2005

Related U.S. Application Data
(60) Provisional application No. 60/630,489, filed on Nov. 22, 2004.

(51) Int. Cl. A61M 5/00

(52) U.S. Cl. 604/9

(57) ABSTRACT

The present invention provides systems and methods for the maintenance of target CSF volumes in the ventricles of a patient's brain. Systems may comprise a mechanism for remote-sensing of CSF volume and/or intra-cranial pressure, a ventricular catheter, a valve and/or pump affixed in the skull and controlled by a microprocessor in response to signals from the sensing device, and an intra-osseous CSF infusion element which may be embedded in the skull near the sagittal suture or at other bone locations, for transport of the CSF removed from the ventricle to the venous system of the brain or elsewhere.

### CROSS-REFERENCES TO RELATED APPLICATIONS

This application claims the benefit of prior provisional application No. 60/630,489 (Attorney Docket No. 025722-000100US), filed on Nov. 22, 2004, the full disclosure of which is incorporated herein by reference.

### BACKGROUND OF THE INVENTION

Hydrocephalus is fundamentally a hydrodynamic disorder characterized by abnormal accumulation of cerebrospinal fluid (CSF) in the brain and spinal column. Normally, CSF is produced within the brain and circulated throughout the subarachnoid space to buoy, cleanse and nourish the brain and spinal cord before being reabsorbed back into the bloodstream. The entire volume of CSF in and around the brain is turned over once every 8 hours in a well-defined dynamic CSF flow pattern. Anything affecting the balance between production, circulation and absorption of CSF leads to significant changes in intra-cranial pressure-volume dynamics. The end result is an increase in intra-cranial pressure (ICP) and/or CSF volume. This abnormal accumulation of CSF is referred to as hydrocephalus. It is the most common neuropathology in infancy and childhood, affecting 1 to 1.5% of the population. Untreated, hydrocephalus is progressive and ultimately fatal.

In theory, hydrocephalus can be treated by decreasing CSF production, improving CSF flow patterns or improving CSF absorption. In practical terms, however, the only successful methods of treating hydrocephalus involve improving flow and absorption patterns by diverting CSF, either internally or externally. For almost fifty years, hydrocephalus has been treated by CSF diversion or “shunting” from the brain to an external absorptive site such as the peritoneal or pleural cavity, the jugular vein or other veins leading centrally, the right atrium, ureter, gall bladder or sub-phrenic space. All of these sites lead into the venous circulation to allow CSF to be re-cycled back into bloodstream, as occurs in the natural setting.

Examples of shunt systems for the continuous drainage of CSF to another part of the body are the Medtronic-PS Medical Delta Shunt and the CSF-Flow Control Shunt Assembly (U.S. Pat. No. 4,560,375), schematized in FIG. 1. In some cases, such as patients with head trauma who may have increased intra-cranial pressure for a short period of time, it is desirable to continuously drain excess CSF to an external device. Prior art examples of this are the Medtronic-PS Medical Becker System and the EDM Drainage System, and patents addressing this approach include U.S. Pat. Nos. 4,731,056 and 5,772,625.

A typical shunt is comprised of a ventricular catheter (or, in some cases, a spinal catheter) a valve and a distal catheter, all connected by a long tube. These shunts tend to over-drain intra-cranial CSF and previous technology fails to recognize the importance of adequate CSF volumes within the cerebral ventricles and subarachnoid spaces. Patients must be continually monitored, and all too frequently these assemblies require surgical maintenance. Shunt systems based on volumetric CSF removal have been proposed in US2003/0004495.

Normally, CSF is absorbed back into the venous system within the nervous system, predominantly into veins or lymphatics located adjacent to the skull. In humans, the majority of CSF absorption occurs along the superior sagittal sinus—especially along the middle third near the vertex of the skull. CSF passes through natural one-way valves called arachnoid granulations located primarily along the sides of the sagittal sinus (lacunae laterals). Mimicking nature, the sagittal sinus itself is the ideal site into which to artificially divert excess or abnormal CSF, but the sagittal sinus has not proven to be safely or directly accessible. A safe alternate route to the superior sagittal sinus would improve CSF diversion systems by simplifying current CSF shunt valve and tubing designs and practices. The current invention uses a previously unexplored anatomical pathway to the venous system of the superior sagittal sinus without directly accessing this critically important anatomical structure.

Current CSF shunts are fraught with problems which may include infection, occlusion, fracture, overdrainage, peritoneal scarring, pulmonary hypertension and glomerulonephritis, regardless of the type of valve and tubing system used. Even ventriculoscopic fenestrations and third ventriculostomy procedures have failed to cure the majority of people with CSF abnormalities. CSF is normally re-absorbed into the superior sagittal sinus along the skull vertex, and a new CSF management system using this natural pathway may avoid many of these complications. Our experiments have proven the existence of an efficient anatomical pathway through the bone of the skull into the venous system of the brain. Intra-osseous infusion devices have been developed and tested and the designs described herein all achieve the desired result of access to the subjacent intra-cranial venous system. The invention refers to new anatomical information and a new intra-osseous strategy for CSF absorption in the treatment of abnormal CSF disorders.

The long distances over which the ventricular catheter and the distal catheter are separated in the prior art is of particular concern. Because patients are routinely mobile, the pressure differential experienced in the drainage system can be quite variable, and changes over an order of magnitude in the space of a few seconds are not uncommon as patients move from a prone to a standing position, for example. This can lead to uncontrolled siphoning and overdrainage of the ventricle. The prior art has attempted to address this issue, with limited success, through the development of valves employing anti-siphoning devices. The current invention eliminates the need for this altogether, by keeping all components of the shunt system in close proximity in the skull.

Another weakness in the prior art is the reliance on intra-cranial pressure as the sole trigger for valve actuation. Prior art systems use valves with a pre-set pressure value, so that one-way flow from the ventricle of the brain to the distal catheter is initiated only when CSF pressure in the brain exceeds that value. The primary problem with this approach is that CSF pressure, per se, does not appear to be the most important factor in maintenance of brain tissue health. Rather, it is the volume of CSF in the ventricles and subarachnoid space that is most critical, and too little fluid volume can be just as deleterious to brain function as excess fluid volume. The current invention senses both intra-cranial pressure and ventricular fluid volume, and activates the drainage pathway only in response to excess CSF levels thereby maintaining appropriate fluid volume in the brain.

### BRIEF SUMMARY OF THE INVENTION

The present invention provides systems and methods for the maintenance of target CSF volumes in the ventricles of a patient's brain. Systems may comprise a mechanism for remote-sensing of CSF volume and/or intra-cranial pressure, a ventricular catheter, a valve and/or pump affixed in the skull and controlled by a microprocessor in response to signals from the sensing device, and an intra-osseous CSF infusion element which may be embedded in the skull near the sagittal suture or at other bone locations, for transport of the CSF removed from the ventricle to the venous system of the brain or elsewhere.

CSF collection sites along the sides of the superior sagittal sinus are preferred based on the relationship of the skull and scalp to the intra-cranial venous system. Infusions through bone (intra-osseous infusion) to the venous system through the skull are preferred so that the entire CSF collection and drainage may be located intra-cranially. Infusion of fluid into the skull bone near the sagittal sinus results in rapid, reliable, remote access to the venous system without the need to manipulate the venous sinus itself. Special intra-osseous infusion devices have been developed for the unique bone of the skull.

The current invention provides a method and system for intra-cranial CSF absorption that mimics the natural CSF absorption system and affords an artificial CSF diversion (shunt) system that is self-contained in the skull region near the site of natural CSF absorption. This obviates the need for long lengths of tubing leading to the central venous system, pleural space or peritoneal cavity. This is achieved, at least in part, by directing intra-osseous infusion of CSF into a target site in bone (osseous target site), such as bone of the skull or other appropriate bony site such as the vertebral column or pelvis to which this invention refers.

### BRIEF DESCRIPTION OF THE DRAWINGS

FIG. 1 is a schematic diagram of a prior art CSF shunt drainage system.

FIG. 2 is a representation of the CSF spaces of the brain and spinal column.

FIG. 3 is a schematic of the anatomical pathway in the skull described in the current invention.

FIG. 4 is a schematic of the anatomical pathway in the skull with the positioning of the intra-osseous catheter superimposed.

FIG. 5 shows the positioning of a typical intra-osseous infusion device of the current invention in the skull for a simple ventricular catheter.

FIG. 6 shows the positioning of a typical intra-osseous infusion device of the current invention in the pelvis for a simple spinal catheter.

FIG. 7 is a vertex view of the current invention device in the skull.

FIG. 8 is a view of an alternative embodiment of the current invention in the skull, showing the pump/valve device and the microprocessor.

FIG. 9 is a drawing of a preferred embodiment of an intra-osseous infusion device for the skull.

FIG. 10 is a drawing of a preferred embodiment of an intra-osseous infusion device for the skull.

FIG. 11 is a drawing of a preferred embodiment of an intra-osseous infusion device for the skull.

FIG. 12 is a drawing of a preferred embodiment of an intra-osseous infusion device for the skull comprising a plenum with a plurality of infusion ports.

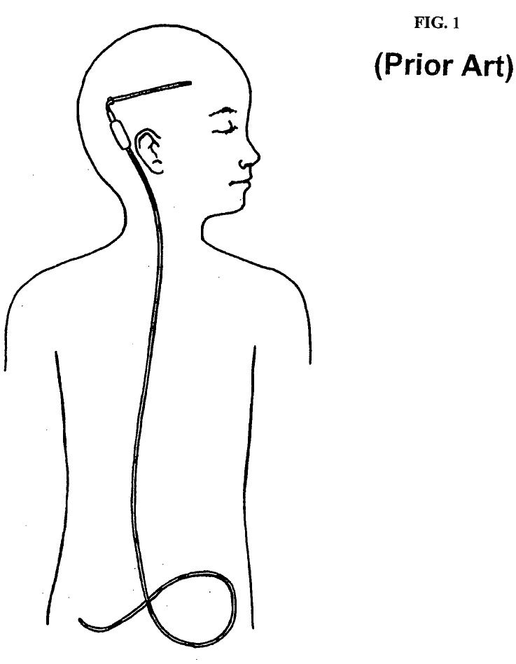

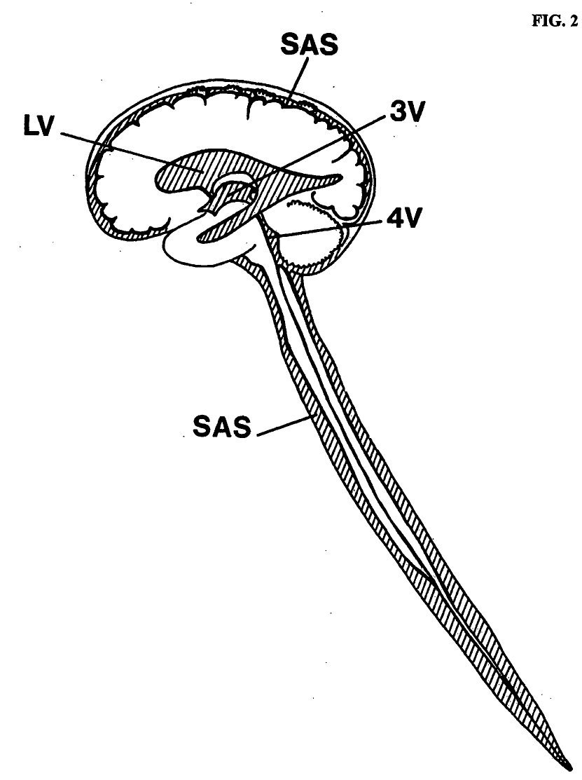

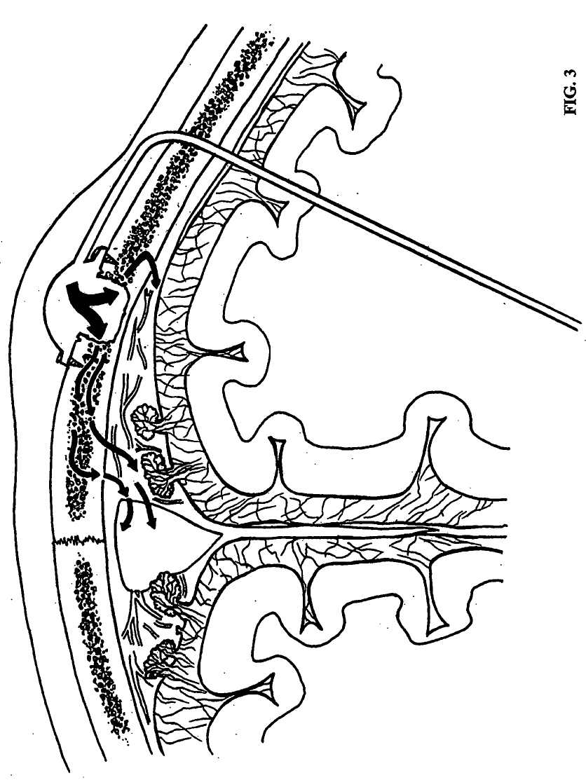

### DETAILED DESCRIPTION OF THE INVENTION

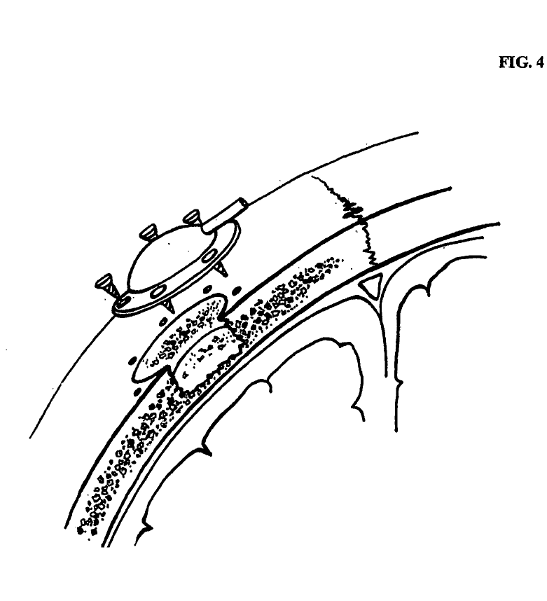

In one embodiment, intra-osseous infusion device 3 of FIG. 4 simply replaces the distal shunt of the prior art, with all other elements remaining unchanged. Thus, the one-way valve leading from the ventricular catheter opens in response to a pressure gradient, and CSF is directed through the intra-osseous infusion device into the skull and thence to the venous system of the brain rather than to other distal sites in the body.

This embodiment eliminates the uncontrolled siphoning and overdrainage common to the prior art, but excess CSF extraction remains dependent on pressure gradients rather than on the more physiologically relevant parameter of CSF volume.

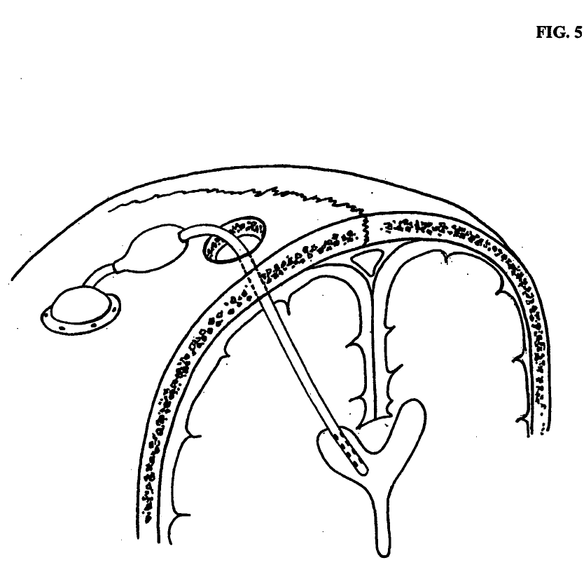

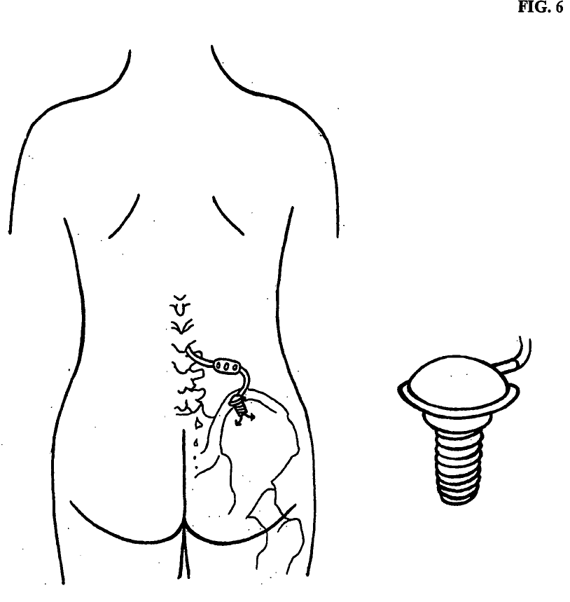

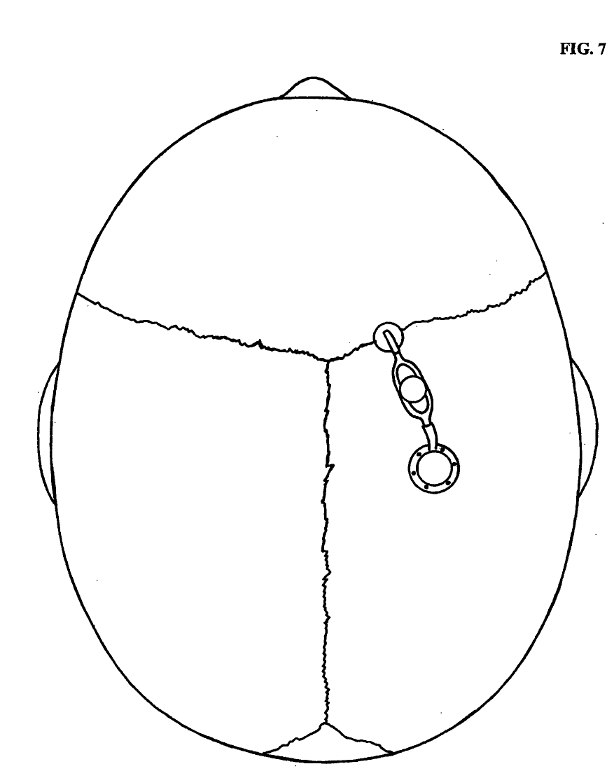

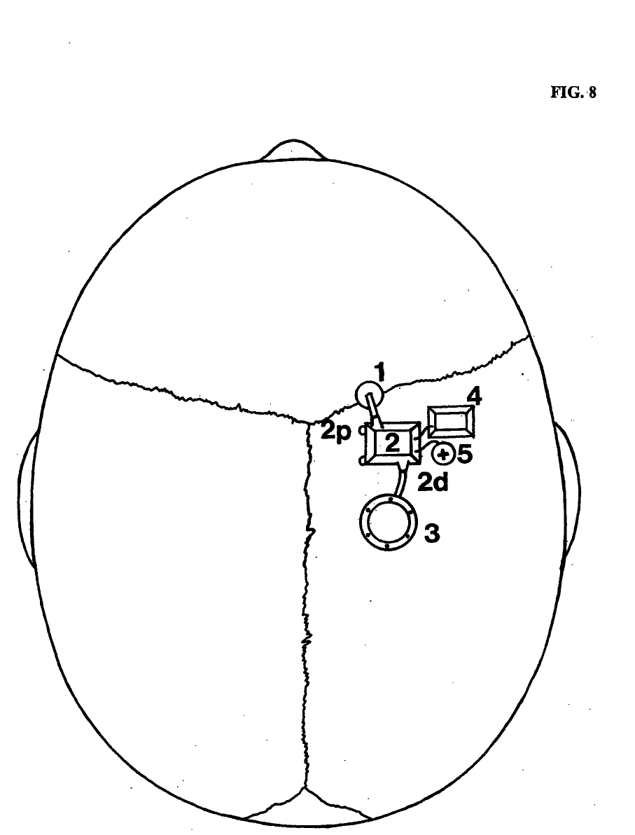

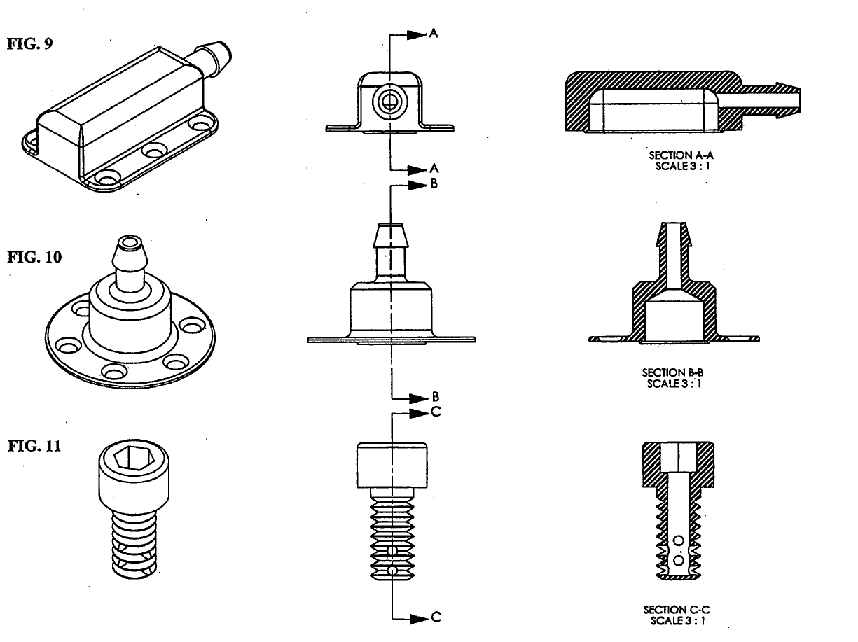

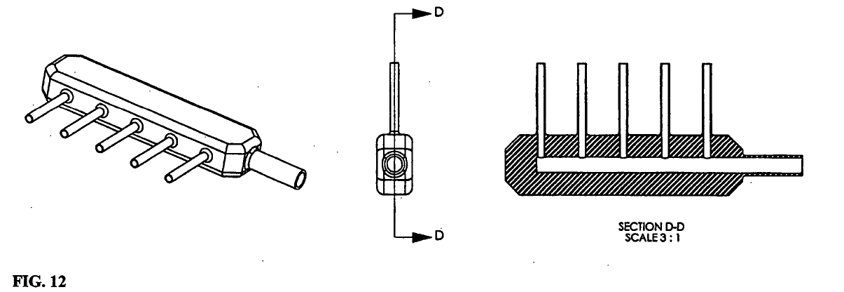

Several embodiments of intra-osseous infusion device 3 are envisioned. A generic design is shown in FIGS. 4-7, and detailed schematics of specific embodiments are provided in FIGS. 8-11. The common attributes are: (1) inlet port(s) to receive CSF from a valve or a pump/valve device, (2) communicating structures (tubes, holes or cavities) to distribute the CSF in the bone to which the device is anchored, (3) means by which the device is sealed into the receiving bone to communicate effectively with the porous bone tissue and to prevent leakage (most commonly, gaskets and cements), and (4) means by which the device is anchored in the bone.

An alternative embodiment of the current invention is shown in situ in FIG. 8. The system includes an implanted catheter 1 leading to a miniature pump/valve 2. This is connected to an intra-osseous infusion device 3. A microprocessor 4 monitors ventricular volume and/or pressure remotely and controls the pump/valve 2 in response to a processed signal indicating excess ventricular fluid volume and/or pressure. The array is powered by a button battery 5.

Remote sensing of ventricular volume is preferred and may be accomplished in many ways, such as by opening proximal valve 2p of pump/valve 2 and querying the ventricular cavity by means of an induced pressure wave generated by pulsatile motion of pump/valve 2. The pressure wave travels down the catheter and into the ventricle, whence it moves out into the CSF space and interacts with the walls of the ventricle. The rebounding pressure waves travel up the catheter where they are detected by an acoustic component (e.g. a piezoelectric membrane) in the pump/valve body. The nature of the pressure waves so detected is interpreted by microprocessor 4 by means of comparison to a calibration table stored onboard in E² memory.

If microprocessor 4 determines that ventricular volume is low or normal, the sensing array enters a period of dormancy, initiating another volume query only following a predetermined time interval (e.g. 15 minutes).

If microprocessor 4 determines that ventricular volume is high, the proximal valve 2p of pump/valve 2 remains open, the distal valve 2d of pump/valve 2 opens, and pump/valve 2 pumps CSF from the proximal catheter to intra-osseous infusion device 3 for a predetermined time (e.g. to deliver 0.5 ml of CSF) whence the fluid enters the bone and ultimately the venous system. At the end of this phase, distal valve 2d closes and another round of querying the ventricle ensues. Depending on the detected volume, the device either initiates another round of pumping to further deplete the excess CSF in the catheterized ventricle, or enters into a dormant state.

In the dormant state the device can either be quiescent (except for the periodic querying of ventricle volume described above) or perform system maintenance. The latter consists of the execution of a programmed series of oscillatory pulses by pump/valve 2 in response to the detection of elevated back-pressure in the distal line. This acts to keep infusion pressures low by clearing microscopic debris from the bone and maintaining hydration of the porous matrix.

A feature of the current invention is the incorporation of self-diagnostic tests that lead to alarm states when conditions deviate from pre-programmed norms. These include, in addition to the detection of a pump malfunction or a low battery state, (1) the detection of unchanging or increasing ICPs and/or ventricular volumes after, for example, three successive iterations of the CSF pumping sequence and (2) the detection of intra-osseous infusion back pressures that exceed pre-set limits. The parameters defining these alarm states are programmable from the surface of the skull, as are those controlling pumping and maintenance cycles. Thus, in an infant with exceedingly large ventricles, it may be desirable to program rapid detection/drainage sequences for the first two months, followed by slower sequences thereafter.

A critical element of the current invention is the placement of the sensing device. This is contained in the pump/valve housing 2 on the surface of the skull rather than implanted in the brain as described by, for example, U.S. Pat. No. 6,731,976 of Penn et al. Implanted sensors are subject to assault by the body's defences and, in the brain, infiltration by the choroid plexus or the ependymal lining, and are notoriously difficult to maintain. Furthermore, malfunctioning implanted sensors are accessible only through surgical intervention. The current invention avoids these problems.

What is claimed is:

1. A method for intra-cranial cerebrospinal fluid (CSF) diversion, said method comprising:
collecting CSF from a ventricle; and
infusing the collected CSF through bone into the venous system.

2. A method as in claim 1, wherein the CSF infusion site is in skull bone.

3. A method as in claim 2, wherein the collected CSF is infused into a site in the skull bone near the sagittal sinus.

4. A method as in claim 1, wherein the CSF infusion site is in vertebral bone or in pelvic bone.

5. A method as in claim 1, wherein the CSF is infused through a plurality of distributed ports into the bone.

6. A method as in claim 1, wherein the CSF is infused through a single port into bone.

7. A method for cerebrospinal fluid (CSF) diversion, said method comprising:
sensing CSF volume in a ventricle;
removing CSF from the ventricle if the CSF volume exceeds a predetermined value.

8. A system for intra-cranial cerebrospinal fluid (CSF) diversion, said system comprising:
a ventricular catheter;
a control element which receives CSF from the ventricular catheter; and
an intra-osseous infusion element which receives CSF from the control element and infuses said CSF into bone tissue.

9. A system as in claim 8, wherein the control element is a valve.

10. A system as in claim 9, wherein the control element is a pressure-responsive valve.

11. A system as in claim 8, wherein the control element is a pump.

12. A system as in claim 8, further comprising a sensor assembly for monitoring ventricular pressure and/or CSF volume.

13. A system as in claim 8, further comprising a controller which controls CSF flow through the control element based on monitored ventricular pressure and/or CSF volume from the sensor assembly.

14. A system as in claim 8, wherein the infusion element comprises a plenum with a plurality of distributed infusion ports.

15. A system as in claim 8, wherein the infusion element comprises a single port.
